(19) United States
(12) Patent Application Publication
(10) Pub. No.: US 2011/0125139 A1
(43) Pub. Date: May 26, 2011

(54) Multi-fiber flexible surgical probe

(76) Inventors: Jack R. Auld, Laguna Niguel, CA (US); Mark H. Farley, Laguna Niguel, CA (US)

(21) Appl. No.: 12/969,212

(22) Filed: Dec. 15, 2010

Related U.S. Application Data
(63) Continuation-in-part of application No. 12/894,721, filed on Sep. 30, 2010.
Application No. 12/894,721 is a continuation of application No. 11/867,302, filed on Oct. 4, 2007, now abandoned.
(60) Provisional application No. 61/305,407, filed on Feb. 17, 2010.

(51) Int. Cl. A61F 9/008

(52) U.S. Cl. 606/4

(57) ABSTRACT

A probe having a flexible, small diameter fiber optic sheathed in a small diameter flexible tube comprising the distal tip of the probe. The small diameters of the fiber and tube allow the fiber to be bent in a tight radius comprising the major portion of the length of the exposed portion of the fiber, with low tube bending forces during insertion, providing a compact design which reduces or eliminates the need for a straight distal portion of flexible tube extending from the cannula. The small diameter tube also allows a greater wall thickness outer cannula to be used, thereby increasing instrument rigidity. One embodiment encompasses a larger flexible tube with corresponding larger bend radius, to encase a plurality of fiber optics, providing separately optimized laser and illumination delivery paths. Anti-friction coating material may be used to further reduce insertion forces.

This application claims priority to U.S. Provisional Application Ser. No. 61/305,407 filed on Feb. 17, 2010 and is a continuation-in-part of U.S. Nonprovisional application Ser. No. 12/894,721 filed on Sep. 30, 2010 which is a continuation of U.S. Nonprovisional application Ser. No. 11/867,302 filed on Oct. 4, 2007, all of which are incorporated herein by reference.

The present invention relates to ophthalmic surgical equipment and more particularly to posterior segment ophthalmic surgical equipment. Even more particularly, the present invention relates to multi-fiber ophthalmic probes.

### BACKGROUND OF THE INVENTION

Microsurgical instruments typically are used by surgeons for removal of tissue from delicate and restricted spaces in the human body, particularly in surgery on the eye, and more particularly in procedures for removal of the vitreous body, blood, scar tissue, or the crystalline lens. Such instruments include a control console and a surgical handpiece with which the surgeon dissects and removes the tissue. With respect to posterior segment surgery, the handpiece may be a vitreous cutter probe, a laser probe, an illumination probe, or an ultrasonic fragmenter for cutting or fragmenting the tissue and is connected to the control console by a long air-pressure (pneumatic) line and/or power cable, optical cable, or flexible tubes for supplying an infusion fluid to the surgical site and for withdrawing or aspirating fluid and cut/fragmented tissue from the site. The cutting, infusion, and aspiration functions of the handpiece are controlled by the remote control console that not only provides power for the surgical handpiece(s) (e.g., a reciprocating or rotating cutting blade or an ultrasonically vibrated needle), but also controls the flow of infusion fluid and provides a source of vacuum (relative to atmosphere) for the aspiration of fluid and cut/fragmented tissue. The functions of the console are controlled manually by the surgeon, usually by means of a foot-operated switch or proportional control.

During posterior segment surgery, the surgeon typically uses several handpieces or instruments during the procedure. This procedure requires that these instruments be inserted into, and removed out of the incision. This repeated removal and insertion can cause trauma to the eye at the incision site. To address this concern, hubbed cannulae were developed at least by the mid-1980s. These devices consist of a narrow tube with an attached hub. The tube is inserted into an incision in the eye up to the hub, which acts as a stop, preventing the tube from entering the eye completely. Surgical instruments can be inserted into the eye through the tube, and the tube protects the incision sidewall from repeated contact by the instruments. In addition, the surgeon can use the instrument, by manipulating the instrument when the instrument is inserted into the eye through the tube, to help position the eye during surgery.

Many surgical procedures require access to the sides or forward portion of the retina. In order to reach these areas, the surgical probes must be pre-bent or must be bendable intra-operatively. Articulating laser/illumination probes are known. See for example, U.S. Pat. No. 5,281,214 (Wilkins, et al.). The articulation mechanism, however, adds extra complexity and expense. One flexible laser probe needing no articulation mechanism is commercially available, but this device uses a relatively large diameter optical fiber sheathed in a flexible tube comprising the distal tip, resulting in a large bend radius and large distal tip diameter with significant bend stiffness. These characteristics require that the distal tip contain a non-bent straight portion for ease of insertion of the bent portion, which must flexibly straighten as it passes through the hubbed cannula. The straight portion of the distal tip allows the bent portion to flexibly pass through the hubbed cannula before the distal cannula of the handpiece enters the hubbed cannula, to allow maximum bending clearance of the flexible portion, thereby minimizing the bending strain and corresponding frictional insertion forces. Such a large bend radius, large diameter flexible tube, and straight distal tip cause the useable portion of the fiber to extend a relatively long distance from the distal tip of the probe and limits laser treatment access of the probe.

A further disadvantage in the known art is the flexibility of the distal cannula, which is a function of the material properties and cross sectional moment of inertia, as determined by the gauge size of the outside diameter of the cannula to fit within the hubbed cannula, and the inside diameter of the cannula to accept the flexible tube. For any given material, the outer and inner diameters of the cannula determine the flexibility of the cannula. This flexibility limits the surgeon's ability to use the instrument to manipulate the position of the eye during surgery.

A further disadvantage in the known art is that it does not offer a non-articulating flexible-tip probe providing both laser and illumination delivery through separate paths optimized for each delivery function. Current surgical procedures require unique delivery patterns for laser and illumination: a narrow beam pattern for laser delivery, and a wide angle pattern for illumination. The optical parameters needed to deliver these two unique patterns differ to the extent that a single delivery path requires either separate instruments or compromised performance of the laser delivery pattern and/or the illumination pattern.

Accordingly, a need continues to exist for a non-articulating flexible-tip probe that does not require a straight portion of flexible tube at the distal tip, and which thus provides a more compact useable tip length, thereby allowing greater laser treatment access to internal posterior structures of the eye without compromising insertion forces. The need also continues to exist for a flexible-tip probe which provides increased rigidity of the distal cannula to facilitate manipulation of the eye position during surgery. In addition, the need exists for a flexible-tip probe which provides both laser and illumination delivery through separate paths optimized for each delivery function.

### BRIEF SUMMARY OF THE INVENTION

The present invention improves upon prior art by providing a probe having a flexible, small diameter fiber within a flexible tube, comprising the non-articulating distal tip of the probe. The small diameter fiber and tube combination allow the fiber to be bent in a tight radius comprising the major portion of the length of the exposed portion of the fiber, minimizing the need for a straight portion to reduce insertion forces. Such a tight radius and compact length allow the fiber greater access to the internal posterior structures of the eye; thus increasing the laser treatment area of the probe, without compromising insertion forces.

Accordingly, an objective of the present invention is to provide a laser probe having a flexible, small diameter non-articulating fiber/tube comprising the distal tip of the probe.

Another objective of the present invention is to provide a laser probe having a flexible, small diameter fiber/tube comprising the distal tip of the probe that is bent in a tight radius comprising the major portion of the length of the exposed portion of the fiber.

A further objective of the present invention is to provide a laser probe that allows greater access to the internal posterior structures of the eye.

A further objective of the present invention is to provide increased rigidity of the distal cannula to facilitate manipulation of the eye position during surgery.

A further objective of the present invention is to provide a flexible-tip laser probe able to deliver both laser and illumination through separate, optimized fiber optic paths.

Other objectives, features and advantages of the present invention will become apparent with reference to the drawings, and the following description of the drawings and claims.

### BRIEF DESCRIPTION OF THE DRAWINGS

FIG. 1 is a perspective view of the probe of the present invention.

FIG. 2 is an elevational view of the probe of the present invention.

FIG. 3 is a cross-sectional view of the probe of the present invention.

FIG. 4 is a cross-sectional view of an alternate embodiment of the present invention, having separate laser and illumination fiber optic delivery paths.

FIG. 5 is a cross-sectional magnified view of distal end of an embodiment of the present invention shown in FIG. 4.

### DETAILED DESCRIPTION OF THE INVENTION

Embodiments of the probe of the present invention provide for a flexible illuminated laser probe with separate, optimized fibers for laser and illumination in a single instrument designed for minimally invasive Trocar-entry surgical systems, unlike the prior art which does not provide for separate fibers to deliver laser and illumination light and that can be used in minimally invasive Trocar-entry surgical systems. The embodiments of this invention thus can provide a probe having optimal illumination intensity, ease of insertion to a surgical site, and a compact tip for broad treatment access. Some of the advantages that can be provided by the embodiments of this invention are: minimally invasive retinal photo-coagulation with directed, optimized illumination of a treatment area; laser and illumination in a single instrument, allowing a surgeon to perform self-scleral depression; compact curved tip and short active length provide broad access to peripheral retina; reduces or eliminates the possibility of elliptical burn associated with straight tipped laser probes; help to avoid lens contact when treating a surgical site opposite to entry port; and facilitate treatment posterior to the sclera buckle.

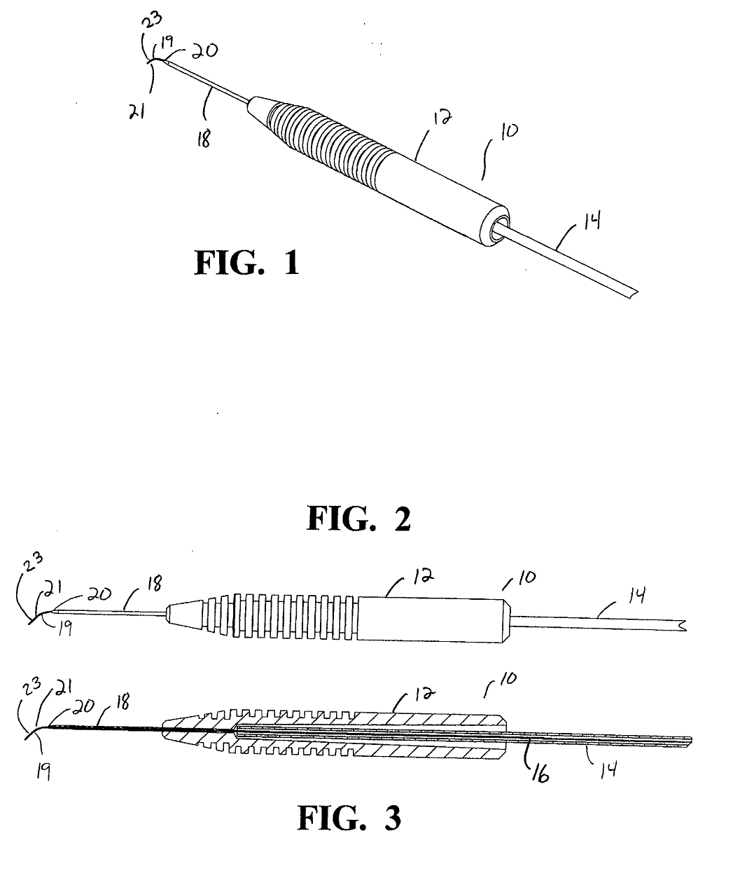

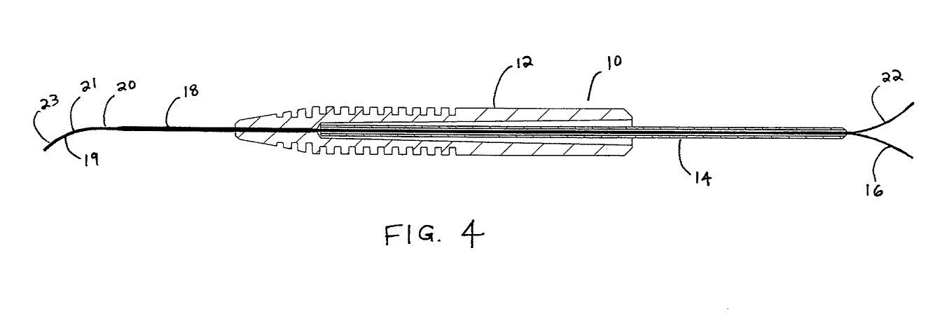

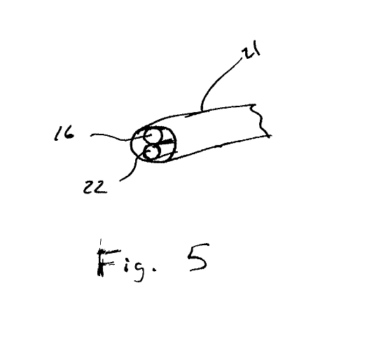

As best seen in the FIGS. 1-5, probe 10 of the present invention generally consists of handle or body 12, containing or encasing a laser fiber optic 16 and/or an illumination fiber optic 22, flexible tube 21, distal cannula 18, and fiber optic sheath 14. Body 12 is generally hollow and can be made from any suitable material such as stainless steel, titanium or thermoplastic. Cannula 18 may be made from any suitable material such as titanium or stainless steel and held within body 12 by any conventional method, such as adhesive or crimping. Fiber optic sheath 14 may be any suitable tubing such as thermoplastic or silicone. In some embodiments, the probe can comprise a plurality of fiber optic cables, each having one or more optical fibers (e.g., fiber optics such as laser fiber optic 16 and illumination fiber optic 22). The plurality of fiber optic cables and fiber optics can have the same or similar optical properties or can each have unique optical properties suitable for their purpose (e.g., illumination or laser light).

Laser fiber optic 16 and illumination fiber optic 22 can be connected on a proximal end (not shown) to any suitable laser or illumination source through a connector of a type well-known in the art and are surrounded by flexible tube 21 with exposed portion 19. Flexible tube 21 is made from a shape memory alloy such as Nitinol, and is held within cannula 18 by any conventional method, such as adhesive or crimping, and encases laser fiber optic 16 and/or illumination fiber optic 22, which are held to inner diameter of flexible tube 21 by any conventional method such as adhesive or crimping. Laser fiber optic 16, illumination fiber optic 22, and exposed portion 19 of flexible tube 21 extend beyond distal end 20 of cannula 18 a distance of approximately 3 millimeters to 14 millimeters, with approximately 4 millimeters to 6 millimeters or 11 millimeters to 13 millimeters being most preferred, respectively for a single fiber optic or a plurality of fiber optics encased in the flexible tube 21.

Laser fiber optic 16 and illumination fiber optic 22 may be made of any fiber optic material suitable for conducting laser or illumination light, respectively. Preferable for a single laser delivery fiber optic is silica (or glass) with an outer diameter of between 100 μm and 125 μm with at least exposed portion 19 of flexible tube 21 being a 33 gauge (approximately 0.008 inches OD) flexible nitinol tube bent at an angle of approximately 30-45° on a radius of approximately between 4.5 millimeters and 6 millimeters along exposed section 19. Importantly, the section of laser fiber optic 16 within exposed section 19 can be curved or bent beginning at or near distal end 20 of cannula 18, with minimal or no straight section near distal end 20 of cannula 18. Such a construction improves peripheral laser treatment access near the point of entry of cannula 18. By virtue of the smaller diameter flexible tube with significantly reduced cross sectional moment of inertia, the simultaneous insertion force of the exposed section 19 with the cannula 18 into a hubbed surgical cannula remains within an optimal range to facilitate manual insertion and extraction.

Preferable material for a laser fiber optic with additional illumination fiber optic, or for a plurality of fiber optics, is silica or plastic or a combination thereof, with outer diameter between 100 μm and 250 μm with at least exposed portion 19 of flexible tube 21 being a 31 to 28 gauge (approximately 0.010 to 0.015 inches OD) flexible nitinol tube bent at an angle of approximately 30-45° on a radius of approximately between 7 millimeters and 15 millimeters along exposed portion 19. Importantly, the section of laser fiber optic 16 and/or illumination fiber optic 22 within exposed section 19 can be curved or bent beginning at or near distal end 20 of cannula 18, with minimal or no straight section near distal end 20 of cannula 18. Such a construction provides both the laser and illumination functions, as well as improved peripheral laser treatment access near the point of entry of cannula 18. By using a minimized flexible tube diameter, bend radius, and straight section, the insertion force of the exposed section 19 into a hubbed surgical cannula remains within an optimal range to facilitate manual insertion and extraction, while providing the additional illumination function. A further reduction of insertion force may be realized by the use of anti-friction coating 23 on the exposed section 19 of flexible tube 21.

In use, exposed section 19 encasing laser fiber optic 16 and/or illumination fiber optic 22 can be straightened so that exposed section 19 can be inserted into an eye through a 23 gauge or a 25 gauge hubbed cannula. Once in the eye, the shape memory characteristics of the nitinol tube cause exposed section 19 to resume its curved configuration.

While certain embodiments of the present invention have been described above, these descriptions are given for purposes of illustration and explanation. Variations, changes, modifications and departures from the systems and methods disclosed above may be adopted without departure from the scope or spirit of the present invention.

We claim:

1. A probe, comprising:
a) a generally hollow body;
b) a cannula attached to the distal end of the body;
c) a plurality of fiber optic cables extending through the hollow body, each of the plurality of fiber optic cables having a fiber optic and extending through the cannula; and
d) an exposed portion of the fiber optics, the exposed portion of the fiber optics extending beyond a distal end of the cannula, the exposed portion of the fiber optics encased in a nitinol tube that is bent along a radius of between approximately 4.5 millimeters and 15.0 millimeters.

2. The probe of claim 1 wherein the nitinol tube is bent at an angle of approximately 30-45 degrees.

3. The probe of claim 1 wherein one or more of the plurality of fiber optics has an outer diameter of between approximately 100 μm and 250 μm.

4. The probe of claim 1 wherein the exposed portion extends beyond the distal end of the cannula a distance of approximately 3.0 millimeters to 8.0 millimeters.

5. The probe of claim 4 wherein the exposed portion extends beyond the distal end of the cannula a distance of approximately 4.0 millimeters to 6.0 millimeters.

6. The probe of claim 1 wherein the exposed portion extends beyond the distal end of the cannula a distance of approximately 8.0 millimeters to 14.0 millimeters.

7. The probe of claim 6 wherein the exposed portion extends beyond the distal end of the cannula a distance of approximately 11.0 millimeters to 13.0 millimeters.

8. The probe of claim 1 wherein the outer diameter of the exposed portion is coated with an anti-friction material.
